(19) United States
(12) Patent Application Publication
(10) Pub. No.: US 2009/0156561 A1
(43) Pub. Date: Jun. 18, 2009

(54) Use of Cholinesterase Inhibitors In Smoking Cessation

(76) Inventor: John C. Seed, Princeton Junction, NJ (US)

Correspondence Address: FOX ROTHSCHILD LLP, 2000 MARKET STREET, 10th Floor, PHILADELPHIA, PA, 19103, US

(21) Appl. No.: 12/140,785

(22) Filed: Jun. 17, 2008

Related U.S. Application Data
(60) Provisional application No. 61/013,424, filed on Dec. 13, 2007.

(51) Int. Cl. A61K 31/662; A61K 31/445; A61K 31/27; A61P 25/34

(52) U.S. Cl. 514/112; 514/139; 514/319; 514/490

(57) ABSTRACT

A method of causing a patient to reduce or cease tobacco use that involves the administration of a cholinesterase inhibitor.

### FIELD OF INVENTION

This invention relates to a method of smoke cessation that involves the administration of an effective amount of a cholinesterase inhibitor to a patient, to help the patient quit smoking. The invention further relates to the use of quasi-irreversible acetylcholinesterase inhibitor, such as an organophosphate in helping a patient quit smoking. The invention further relates to the use of a combination of cholinesterase inhibitors in helping a patient quit smoking.

### BACKGROUND OF THE INVENTION

Tobacco smoking continues to be the leading cause of preventable death in the world. It is estimated that tobacco use causes nearly 5 million deaths per year, and that by year 2020, tobacco use will cause more than 10 million deaths annually. Of the approximately 440,000 deaths per year in the United States, about 40,000 are the result of secondhand smoke exposure.

Tobacco-related costs are staggering. The Center for Disease Control estimates that in the United States, cigarette smoking costs more than $167 billion, based on lost productivity ($92 billion) and health care expenditures ($75 billion). Health care costs associated with exposure to secondhand smoke averages about $10 billion annually.

Most smokers are aware of these medical problems, early deaths, and health and other financial costs. Indeed, among adult smokers in the United States, about 70% report that they want to quit smoking completely, and about 40% try to quit each year. Many smokers find it difficult to quit smoking, even in face of serious smoking-related diseases in themselves or close family members or friends. Due to the highly addictive nature of tobacco, most smoking cessation methods have a very poor success rate. Approximately only 3% of smokers succeed in quitting smoking using will power alone; nicotine replacement therapy doubles this to approximately 6%.

Because it is so desirable, yet difficult to quit smoking, there is a great need for an efficacious method to help smokers quit.

### SUMMARY OF THE INVENTION

One embodiment of this invention is a method of aiding a patient to reduce or cease tobacco use that comprises administering to the patient a therapeutically effective amount of a quasi-irreversible cholinesterase inhibitor.

Another embodiment of this invention is a method of aiding a patient to reduce or cease tobacco use that comprises administering to the patient a therapeutically effective amount of at least two reversible cholinesterase inhibitors.

This invention is quite effective in discouraging smokers from smoking since it induces certain very uncomfortable physiological effects in the patient when the patient is exposed to tobacco smoke.

### DETAILED DESCRIPTION OF THE INVENTION

The majority of smokers have great difficulty in quitting smoking. This is true even if a smoker is well aware of smoking's dangers. Many smokers may be so addicted to smoking that they smoke even having serious smoking-related diseases. The immediate beneficial feelings that a smoker gets from smoking, such as, feelings of satisfaction, stress relief, even euphoria, appears to outweigh the known long-term costs. The present invention addresses this immediate-term benefit vs. long-term costs balance by mitigating the immediate-term benefit. The present invention is a method of treating the smoking habit by mitigating the immediate-term benefit by replacing the beneficial feelings with negative physical feelings. The physical feelings are so negative to mask any beneficial feelings, or to replace the beneficial feelings. The physical feelings occur each and every time that the smoker inhales tobacco smoke while on therapy.

The negative physical feelings occur only upon exposure of a patient to tobacco smoke. One embodiment of the present invention is an administration of a substance to patient who is attempting to quit smoking, wherein the substance produces negative feelings in the patient upon exposing the patient to tobacco smoke. The substance that produces negative feelings in the patient is a cholinesterase inhibitor.

A patient may experience sickness or discomfort to a such a degree, that the patient will not smoke even if the smoker decides not to stop smoking after starting therapy or wants to increase exposure to the tobacco smoke.

Under one embodiment the negative physical feeling felt by a patient upon exposure to tobacco smoke is not severe enough to cause sickness or discomfort of a great magnitude, but is simply a feeling of unease which is sufficient to remind the patient not to smoke.

Under one embodiment of the present invention, the negative physical feelings are feelings of sickness or discomfort. This discomfort may be action of the parasympathetic nervous system, causing bradycardia, hypotension, hypersecretion, bronchoconstriction, GI tract hypermotility, and decrease of intraocular pressure. This discomfort may be the exhibition of salivation, lacrimation, urination, or defecation.

Some of the major effects of anticholinesterases include actions on the autonomic nervous system (i.e., parasympathetic nervous system will cause bradycardia, hypotension, hypersecretion, bronchoconstriction, GI tract hypermotility, and decrease intraocular pressure), SLUD syndrome, and actions on the neuromuscular junction resulting in prolonged muscle contraction. SLUD syndrome (salivation, lacrimation, urination, and defecation) is a syndrome of pathological effects indicative of massive discharge of the parasympathetic nervous system.

The present method of aiding smoking cessation in a patient is especially useful in cases where the smoking habit is very strong, such as in cases where the patient tried to quit several times previously, but was not successful in doing so. The present method is also especially useful in patients who are incapable of formulating or holding the desire to stop smoking, such as patients who are experiencing absentmindedness, forgetfulness, mental illness, dementia or insanity, or are not willing or able to control their actions. The present method is also useful in patients who put others at great risk by smoking, such as in highly flammable environments, explosive environments, clean rooms, oxygen tents, or other places where the hazards of smoking are very great.

The present method of aiding smoking cessation is also useful to patients who are not habitual smokers, to patients who show mild or little addiction, and to patients who have little or only a moderate amount of difficulty in quitting smoking.

The phrases “smoking cessation” and “quitting smoking” as used herein means the effort to reduce or stop smoking tobacco products. This decrease or stopping may be temporary or permanent. This decrease or stopping may be for the duration of time that it takes for the body to regenerate cholinesterase, but preferably for the life of the patient.

The phrase “aiding tobacco use cessation” or “aiding of tobacco use cessation” means that the prescribed method of treatment is helpful to the patient in ceasing to use tobacco, including but not limited to smoking. The definition of aiding smoking cessation includes methods where the method of treatment is the sole method used to make the patient quit smoking. The definition of aiding smoking cessation includes methods where the method of treatment is the main method used to make the patient quit smoking in concert with other methods. Furthermore, the definition of aiding smoking cessation includes methods where the method of treatment is a secondary or auxiliary or tangential method used to make the patient quit smoking in support of other methods.

Tobacco products include any number of nicotine-containing products. This includes cigarettes, pipe tobacco or cigars. Tobacco products also include products which contain nicotine, and which acts as cigarettes, pipes or cigars, but which are not necessarily based on tobacco plants. Tobacco products include snuff, chewing tobacco, snus, dokha, creamy snuff, gutka, and tobacco water.

The phrase “effective amount” or the “effective dosage” is the amount or dosage which produces the desired effect in aiding smoking cessation. The effective amount or effective dosage is patient dependent and goal dependent. For those patients who need immediate-term benefit vs. long-term costs is heavily in favor of the former (such if the patient's habit is very strong, or the patient has a high probability of facing a serious smoking disease) then the effective amount or effective dosage should be high. High dosage is indicated by incapacitative abdominal pain upon tobacco exposure. In other patients or other cases, the effective amount or the effective dose refers to the amount which causes only a mild or moderate discomfort.

It has been recognized that sub-lethal exposure of nerve gas on patient destroys the cholinesterase enzyme in the patient's body. It takes about one month for the body to build up new cholinesterase. The nerve gas cholinesterase inhibitors bind to the enzyme and inhibit the enzyme from functioning, but they do not chemically bind to a part of the enzyme that blocks any activity it has against acetylcholine.

Under one of the preferred embodiments of the invention, the active ingredient is selected from one of several quasi-irreversible acetylcholinestarase inhibitors. The use of quasi-irreversible acetylcholinestarase inhibitors destroys cholinesterase and this sensitizes the gastrointestinal (GI) tract to the effects of nicotine. The effective dosage of the sensitization of the GI tract is about the same as the dose level that produces mild constriction of the pupil. The sensitization of the GI tract is such that one puff on a cigarette results in such abdominal pain that a person will experience severe intestinal cramps. The cramps disappear in 20 to 30 minutes. The sensitization of the GI tract lasts for about 3 to 5 weeks. There are no long term side effects such as diarrhea, constipation, belching, passing flatus, weight loss, weight gain, or loss. Being in a room with others who smoke, causes immediate mild to moderate cramps. Loss of appetite occurs as long as the patient does not smoke.

The present method of aiding tobacco use cessation in a patient comprises administering to the patient a therapeutically effective amount of an acetylcholinesterase inhibitor. Under the preferred embodiment acetylcholinesterase inhibitor is delivered in any one of several methods, including orally, parenterally, intravenously, or topically. In any of these medicaments the acetylcholinesterase inhibitor is the active ingredient.

A “acetylcholinestarase inhibitor” is a substance, a compound or a mixture of compounds that inhibit the cholinesterase enzyme from breaking down acetylcholine, so increasing both the level and duration of action of the neurotransmitter acetylcholine.

An “acetylcholinesterase” is a type of a cholinesterase. It is also known as red blood cell cholinesterase, erythrocyte cholinesterase, or acetylcholine acetylhydrolase. It is found primarily in the blood and neural synapses.

An “acetylcholine” is 2-acetoxy-N,N,N-trimethylethanaminium ion. It is a neurotransmitter in both the peripheral nervous system (PNS) and central nervous system (CNS) in many organisms including humans. Acetylcholine is one of many neurotransmitters in the autonomic nervous system (ANS) and the only neurotransmitter used in the somatic nervous system. It is also the neurotransmitter in all autonomic ganglia.

A “cholinesterase” is an enzyme that catalyzes the hydrolysis of the neurotransmitter acetylcholine into choline and acetic acid. Such a reaction is necessary to allow a cholinergic neuron to return to its resting state after activation.

A “reversible cholinesterase inhibitor” is an inhibitor which functions as reversible competitive or noncompetitive inhibitors of cholinesterase. Reversible cholinesterase inhibitors include organophosphates, carbamates, phenanthrine derivatives, piperidines, Tacrine, tetrahydroaminoacridine, Edrophonium, and phenothiazines. Organophosphates include substances such as metrifonate. Carbamates include substances such as physostigmine, neostigmine, pyridostigmine, ambenonium, demarcarium, and rivastigmine. Phenanthrine derivatives includes galantamine. Piperidines includes substances such as donepezil and E2020.

A “quasi-irreversible cholinesterase inhibitor” is a substance or a compound which functions as quasi-irreversible inhibitors of cholinesterase. Such compounds are those most likely to have use as chemical weapons or pesticides. Quasi-irreversible acetylcholinestarase inhibitors include organophosphates and carbamates. Organophosphates includes substances such as echothiophate, diisopropyl fluorophosphate, cyclosarin, soman, tabun, VX, VE, VG, VM, diazinon, malathion, and parathion. Carbamates include aldicarb, bendiocarb, bufencarb, cabaryl, carbendazim, carbofuran, chlorbufam, chloropropham, ethiofencarb, formetanate, methiocarb, methonyl, oxamyl, phenmedipham, pinmicarb, pirimicarb, propamocarb, propham, and propoxur.

The definition of a “cholinesterase inhibitor” also includes a nerve agent. Nerve agents include GA, tabun, GB, sarin, GD, soman, GF, cyclosarin, GV, VE, VG, VM, and VX.

Under a more preferred embodiment, the quasi-irreversible acetylcholinestarase inhibitor is a organophosphate. Under a still more preferred embodiment the organophosphate is tabun.

Under another preferred embodiment the quasi-irreversible acetylcholinestarase inhibitor is selected from commercially available sources. An example of commercially available quasi-irreversible acetylcholinestarase inhibitor is diisopropylfluorophosphate. Diisopropylfluorophosphate has been marketed as a 0.025% ophthalmic ointment by Merck & Co. Inc. under the trade name Floropryl with the generic name isoflurophate. As with other quasi-irreversible acetylcholinestarase inhibitors, no long-term adverse effects have been observed with high dosage of diisopropylfluorophosphate.

Under another embodiment of the present invention a method of aiding smoking cessation in a patient comprises administering to the patient a therapeutically effective amount of a combination of at least two different reversible cholinesterase inhibitors. Under one embodiment the first of the two cholinesterase inhibitors is an acetylcholinesterase-selective inhibitor, and the second of the two cholinesterase inhibitors is a combined butyrylcholinesterase and acetylcholinesterase inhibitor. Under a more preferred embodiment the first cholinesterase inhibitor is donepezil, and the second cholinesterase inhibitor is rivastigmine.

It is surprising that administering to a patient a single reversible cholinesterase inhibitor does not illicit the effect of negative physical feelings upon exposure to tobacco smoke. Administration of either donepezil or rivastigmine does not appear to aid in smoking cessation programs.

The term “donepezil” refers to a drug comprising as an active ingredient 2-[(1-benzyl-4-piperidyl)methyl]-5,6-dimethoxy-2,3-dihydroinden-1-one, a pharmaceutically acceptable salt, or a solvate, or a hydrate, or a prodrug, thereof. Donepezil is a reversible acetylcholinesterase inhibitor. The commercially available form of donepezil is marketed under the trade name Aricept®, which is a well known medicament used in a treatment of Alzheimer's Disease. Donepezil is postulated to exert its therapeutic effect by enhancing cholinergic function. This enhancement is accomplished by increasing the concentration of acetylcholine through reversible inhibition of its hydrolysis by acetylcholinesterase. Donepezil is selective for acetylcholinesterase.

The term “rivastigmine” refers to a drug comprising as an active ingredient (S)-3-[1-(dimethylamino)ethyl]phenyl ethylmethylcarbamate, a pharmaceutically acceptable salt, or a solvate, or a hydrate, or a prodrug, thereof. Rivastigmine is a cholinergic agent. The commercially available form of rivastigmine is marketed under the trade name Exelon®, which is a well known medicament used in a treatment of Alzheimer's Disease. Rivastigmine is a cholinesterase inhibitor that inhibits both butyrylcholinesterase and acetylcholinesterase.

The term “prodrug” refers to a form of the compound of the drug suitable for administration to a patient without undue toxicity, irritation, allergic response, and the like, and effective for their intended use, including ketal, ester and zwitterionic forms. A prodrug is transformed in vivo to yield the parent compound of the above formula, for example by hydrolysis in blood. A thorough discussion is provided in T. Higuchi and V. Stella, Pro-drugs as Novel Delivery Systems, Vol. 14 of the A. C. S. Symposium Series, and in Edward B. Roche, ed., Bioreversible Carriers in Drug Design, American Pharmaceutical Association and Pergamon Press, 1987, both of which are incorporated herein by reference.

The term “solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. Solvate encompasses both solution-phase and isolable solvates. Exemplary solvates include ethanolates, methanolates, and the like. The term “hydrate” refers to a solvate wherein the solvent molecule(s) is/are H₂O.

Another embodiment of the present invention is a treatment regimen to aid smoking cessation. Such a regimen comprising the steps of:
(a) administering to a patient a low dosage rivastigmine patch for a week;
(b) administering to the patient a high dosage rivastigmine patch for a second week; and
(c) administering to the patient a high dosage rivastigmine patch and donepezil for subsequent weeks.

An example of the low dosage rivastigmine patch is an Exelon Patch (4.6 mg). An example of the high dosage rivastigmine patch is an Exelon Patch (9.5 mg). An example of donepezil is Aricept (5 mg, once per day). During the weeks when the patient takes both the rivastigmine and donepezil the patient is not able to smoke due to unpleasant feelings upon exposure to smoke. In cases when the unpleasant feelings are not strong enough to overcome the need to stop smoking, the dosage of the donepezil should be increased, so that unpleasant feelings are experienced by the patient. Under the preferred embodiment, this regimen is to be followed until the patient does not experience the need to smoke. It is likely that the regimen is to be followed for about a year, or in some cases for the rest of the patient's life.

If two acetylcholinesterase-selective inhibitors are used together in the practice of this invention (e.g., a acetylcholinesterase-selective inhibitor and a combined butyrylcholinesterase and acetylcholinesterase inhibitor as described above, the two inhibitors can be formulated into a single unit dosage form. A unit dose can be made by adding a pharmacologically effective amount of each the two agents to a pharmaceutically acceptable carrier. Pharmaceutically acceptable carriers for oral administration include capsules, tablets, pills, powders, troches and granules. In such solid dosage forms, the carrier can comprise at least one inert diluent such as sucrose, lactose or starch. Such carriers can also comprise, as is normal practice, additional substances other than diluents, e.g., lubricating agents such as magnesium stearate.

Pharmaceutically acceptable carriers for rectal administration are preferably suppositories which may contain, in addition to the two inhibitors, excipients such as cocoa butter or a suppository wax.

Effective amounts of the compounds of the present invention can be administered to a subject by any one of several methods, for example, orally as in capsules or tablets, parenterally in the form of sterile solutions or suspensions, and in some cases intravenously in the form of sterile solutions, or topically. Preferred methods of delivery are topical solution and tablets/capsules.

The compounds of the present invention, while effective themselves, can be formulated and administered in the form of their pharmaceutically acceptable addition salts for purposes of stability, convenience of crystallization, increased solubility, and the like. Preferred pharmaceutically acceptable addition salts include salts of mineral acids, for example, hydrochloric acid, sulfuric acid, nitric acid, and the like; salts of monobasic carboxylic acids, for example, acetic acid, propionic acid, and the like; salts of dibasic carboxylic acids, for example, maleic acid, fumaric acid, and the like; and salts of tribasic carboxylic acids, such as carboxysuccinic acid, citric acid, and the like.

Effective quantities of the compounds useful in the practice of this invention can be administered orally, for example, with an inert diluent or with an edible carrier. They can be enclosed in gelatin capsules or compressed into tablets. For the purposes of oral therapeutic administration, compounds of the invention can be incorporated with an excipient and used in the form of tablets, troches, capsules, elixirs, suspensions, syrups, wafers, chewing gums, and the like. These preparations should contain about 0.0001% to 0.1% of active compound of the invention, but can be varied depending upon the particular form. Preferred compositions and preparations according to the present invention are prepared so that an oral dosage unit form contains between 0.0001 and 10 milligrams of the active compound per kg of patient.

Tablets, pills, capsules, troches, and the like can also contain the following ingredients: a binder, such as microcrystalline cellulose, gum tragacanth, or gelatin; an excipient, such as starch or lactose; a disintegrating agent such as alginic acid, Primogel, corn starch, and the like; a lubricant such as magnesium stearate or Sterotes; a glidant such as colloidal silicon dioxide; and a sweetening agent such as sucrose; or saccharin, or a flavoring agent, such as peppermint, methyl salicylate, or orange flavoring. When the dosage unit form is a capsule, it can contain, in addition to materials of the above type, a liquid carrier such as a fatty oil. Other dosage unit forms can contain various materials that modify the physical form of the dosage unit, for example, as coatings. Thus, tablets or pills can be coated with sugar, shellac, or other enteric coating agents. A syrup can contain, in addition to the active compounds, sucrose as a sweetening agent and certain preservatives, dyes, colorings, and flavors. Materials used in preparing these various compositions should be pharmaceutically pure and non-toxic in the amounts used.

For the purpose of parenteral therapeutic administration, the active compound of the invention can be incorporated into a solution or suspension. These preparations should contain at least 0.001% of active compound, but can be varied between 0.0001 and about 5% of the weight thereof. The amount of active compounds in such compositions is such that a suitable dosage will be obtained. Preferred compositions and preparations according to the present invention are prepared so that a parenteral dosage unit contains between 0.01 to 100 milligrams of active compound.

Solutions or suspensions can also include the following components: a sterile diluent, such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, or phosphates, and agents for the adjustment of tonicity such as sodium chloride or dextrose. The parenteral preparation can be enclosed in ampules, disposable syringes, or multiple dose vials made of glass or plastic.

The examples below are illustrative of this invention and are not intended to limit it.

### EXAMPLE 1

A patient, a healthy male habitual smoker of about 20 years of age, was inadvertently exposed to a sublethal dosage of tabun. Shortly upon resuscitation of the patient by medical personnel, the patient pulled out a cigarette, lit it, only to drop to the ground in shock, with blood pressure 60/00. Subsequent resuscitation via intravenous administration of atropine lead to a full recovery of the patient. However, it was surprising to find that the patient had extreme aversion to smoking for about one month after the exposure to tabun. During the one-month period, the patient felt sick if he was in the same room with anyone who smoked; he felt sick if he was down wind about 5 meters from someone who smoked. The primary response to exposure to tobacco smoke was the evacuation of the colon.

### EXAMPLE 2

A mixture of rivastigmine and donepezil was administered to a patient, who was a healthy male, habitual smoker of about 20 years of age. The patient did not appreciate that administration of this mixture to his body would have any effect on his smoking activities. Upon taking a puff on a cigarette, he became nauseous, and had to run to the toilet, where he evacuated his colon.

### EXAMPLE 3

Near lethal dosages of diisopropylfluorophosphate was administered to 27 canines on a daily basis for a period of 1 to 2 years. None of the canines showed any irreversible changes in the central nervous system nor any other organs.

### EXAMPLE 4

PY, a 76 year old male patient, was a habitual smoker who tried unsuccessfully to quit smoking. In order to aid smoking cessation, a combination regimen of 5 mg of donepezil and 1.5 mg rivastigmine, administered once per day, was instituted. The patient was advised that if he started to smoke (taking two or three puffs of a cigarette), he would have to move his bowels. The patient was seen every two weeks for a period of 12 months. He has not smoked since starting on the combination regimen.

### COMPARATIVE EXAMPLE

NS, a 60 year old male patient, was a habitual smoker. In order to aid smoking cessation, 5 mg of donepezil was administered on daily basis for a period of one year. Although the patient moved his bowels more frequently, the effect was not strong enough to aid smoking cessation. The patient continued to smoke.

### EXAMPLE 5

A 63 year old male patient was an occasional smoker. He had been on a combination regimen of donepezil (5 mg, once a day) and rivastigmine (1.5 mg, twice a day), and was not smoking. When he was offered a cigarette, he took two puffs and had to run to the bathroom and barely made it in time. He never smoked again.

### EXAMPLE 6

An exemplary program for aiding smoking cessation, as presented to the patient, is as follows.

(1) apply Exelon Patch (4.6 mg) patch to skin with no hair on it, for instance under the collar bone and leave it there for 24 hours. The next day take the patch off, and apply another one at a different site. The patient may take a shower or swim with the patch on. Apply one patch a day for one week.

(2) After one week, switch to Exelon Patch 9.5 mg.

(3) Start to take donepezil (5 mg, once per day, upon waking up) after the first week of taking Exelon Patch 9.5 mg.

(4) The patient should try to stop smoking once he starts taking donepezil. Smoking should make him feel uncomfortable in his abdomen. If it does not make the patient feel uncomfortable, he should consult his doctor if he needs to increase the dosage of donepezil to 10 mg once a day. If the patient gets into a situation in which he feels he needs to smoke despite the fact that he has been taking these medicines, he could take one loperamide 2 mg tablet, a non-prescription medicine. He would be able to smoke 15-30 minutes after he took loperamide and he should not have any abdominal discomfort for about 4 hours.

(5) The regimen should be continued for about a year, or until the patient no longer desires to use tobacco products.

The present invention may be embodied in other specific forms without departing from the spirit or essential attributes thereof.

I claim:

1. A method of aiding a patient to reduce or cease tobacco use, comprising administering to a patient a therapeutically effective amount of quasi-irreversible cholinesterase inhibitor.

2. The method of claim 1 wherein the quasi-irreversible cholinesterase inhibitor is an organophosphate.

3. The method of claim 2 wherein the organophosphate is a nerve agent.

4. The method of claim 3 wherein the nerve agent is selected from the group consisting of cyclosarin, sarin, soman, tabun, VX, VE, VG, and VM.

5. The method of claim 3 wherein the nerve agent is tabun.

6. The method of claim 2 wherein the quasi-irreversible cholinesterase inhibitor is a diisopropylfluorophosphate.

7. A method of aiding a patient to reduce or cease tobacco use, comprising administering to a patient a therapeutically effective amount of at least two reversible cholinesterase inhibitors.

8. The method of claim 7, wherein first of the two reversible cholinesterase inhibitors is an acetylcholinesterase inhibitor, and the second of the two reversible cholinesterase inhibitors is a cholinesterase inhibitor that inhibits butyrylcholinesterase and acetylcholinesterase.

9. The method of claim 8, wherein the acetylcholinesterase inhibitor is donepezil.

10. The method of claim 8, wherein the cholinesterase inhibitor that inhibits butyrylcholinesterase and acetylcholinesterase is rivastigmine.

11. The method of claim 8, wherein the acetylcholinesterase inhibitor is donepezil, and the cholinesterase inhibitor that inhibits butyrylcholinesterase and acetylcholinesterase is rivastigmine.

12. A method of aiding a patient to reduce or cease tobacco use, comprising the steps of:
(a) administering to a patient a low dosage rivastigmine patch for a week;
(b) administering to the patient a high dosage rivastigmine patch for a second week; and
(c) administering to the patient a high dosage rivastigmine patch and donepezil in subsequent weeks.
